(19) United States
(12) Patent Application Publication
(10) Pub. No.: US 2021/0366007 A1
(43) Pub. Date: Nov. 25, 2021

(54) INFORMATION PROCESSING APPARATUS AND NON-TRANSITORY COMPUTER READABLE MEDIUM

(71) Applicant: FUJIFILM BUSINESS INNOVATION CORP., Tokyo (JP)

(72) Inventors: Akihiro ISHII, Kanagawa (JP); Yusuke TAGUCHI, Kanagawa (JP); Hiroyuki MITSUHASHI, Kanagawa (JP); Shuhei TODA, Kanagawa (JP)

(73) Assignee: FUJIFILM BUSINESS INNOVATION CORP., Tokyo (JP)

(21) Appl. No.: 17/116,616

(22) Filed: Dec. 9, 2020

(30) Foreign Application Priority Data
May 25, 2020 (JP) 2020-090323

(51) Int. Cl. G06Q 30/02; G16H 10/20; G16H 40/67; A61B 5/16

(52) U.S. Cl. CPC G06Q 30/0282; G16H 10/20; G06F 3/0482; G16H 40/67; A61B 5/165; G06Q 30/0203

(57) ABSTRACT

An information processing apparatus includes a processor configured to acquire biological information on a user while the user is using the apparatus, perform control so as to display, on a display section, a rating screen that is used to perform an operation to give a rating related to use of the apparatus, and perform control so as to display, on the display section, different rating screens in accordance with a psychological state of the user indicated by the biological information.

### CROSS-REFERENCE TO RELATED APPLICATIONS

This application is based on and claims priority under 35 USC 119 from Japanese Patent Application No. 2020-090323 filed May 25, 2020.

### BACKGROUND

### (i) Technical Field

The present disclosure relates to an information processing apparatus and a non-transitory computer readable medium.

### (ii) Related Art

Users who used a device or a service are occasionally requested to rate the device or the service. For example, Japanese Unexamined Patent Application Publication No. 2007-87119 describes inquiring of a user about operability rating information that indicates the operability for and the level of satisfaction with user operations. Japanese Unexamined Patent Application Publication No. 2019-32722 describes making display for asking a user to enter a review for an application to be rated in the case where the content of predetermined data collected when the application to be rated operates matches a rating condition that prescribes whether such data are preferable for the user. In addition, there is known a technique of recognizing the state of a user when he/she operates a device. For example, Japanese Unexamined Patent Application Publication No. 2017-170647 describes acquiring biological information on a user who performs an operation using an operation screen and registering information on the operation screen and the biological information on the user in association with each other.

### SUMMARY

In the case where a device requests a user of a service provided by the device itself to rate the service, the device occasionally displays a rating screen that is used for an operation to give a rating. However, giving a rating is an option for the user, and also takes time. Therefore, if a rating screen determined in advance is displayed irrespective of a psychological state such as whether or not the user is satisfied with the subject that he/she used, the user may feel annoyed at performing an operation to give a rating, and not take the trouble to give a rating.

Aspects of non-limiting embodiments of the present disclosure relate to displaying a rating screen related to use of a device that matches the psychological state of a user of the device.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided an information processing apparatus including a processor configured to acquire biological information on a user while the user is using the information processing apparatus, perform control so as to display, on a display section, a rating screen that is used to perform an operation to give a rating related to use of the information processing apparatus, and perform control so as to display, on the display section, different rating screens in accordance with a psychological state of the user indicated by the biological information.

### BRIEF DESCRIPTION OF THE DRAWINGS

Exemplary embodiments of the present disclosure will be described in detail based on the following figures, wherein:

FIG. 1 illustrates an example of the configuration of an image processing system according to an exemplary embodiment;

FIG. 2 illustrates an example of the configuration of an image processing apparatus;

FIG. 3 is a sequence chart illustrating an example of operation performed by the image processing system;

FIG. 4 illustrates an example of screen transition of a display section;

FIG. 5 illustrates an example of screen transition of the display section;

FIG. 6 illustrates a high-rating rating screen according to a first exemplary embodiment;

FIG. 7 illustrates an improvement requesting rating screen according to the first exemplary embodiment;

FIG. 8 illustrates a high-rating rating screen according to a second exemplary embodiment;

FIG. 9 illustrates an improvement requesting rating screen according to the second exemplary embodiment;

FIG. 10 illustrates an example of the level of satisfaction of a user during processes performed while a copy function is activated according to a modification;

FIG. 11 illustrates an example of a high-rating rating screen prepared and displayed in the example illustrated in FIG. 10;

FIG. 12 illustrates an example of the level of satisfaction of a user during processes performed while a scan function is activated according to the modification;

FIG. 13 illustrates an example of an improvement requesting rating screen prepared and displayed in the example illustrated in FIG. 12;

FIG. 14 illustrates an example of a high-rating rating screen according to a modification; and

FIG. 15 illustrates an example of improvement requesting rating screens according to the modification.

### DETAILED DESCRIPTION

1. Configuration

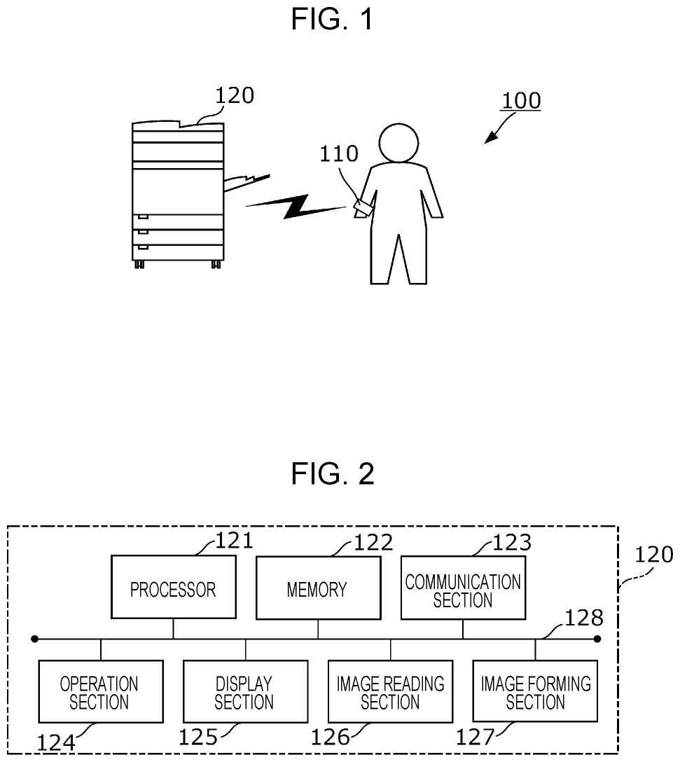

FIG. 1 illustrates an example of the configuration of an image processing system 100 according to the present exemplary embodiment. The image processing system 100 requests a user for a rating related to use of an image processing apparatus 120 after use of the image processing apparatus 120, in order to improve the operability and the functionality of the image processing apparatus 120. This rating is also called “review”, and includes determining whether a subject is good or bad and giving one's opinion, impression, and view about the subject. At this time, the image processing system 100 varies a rating screen that is used to perform an operation to give a rating in accordance with the psychological state of the user. The image processing system 100 includes a wearable terminal 110 and the image processing apparatus 120. The image processing apparatus 120 is an example of the information processing apparatus according to the present disclosure.

The wearable terminal 110 is mounted to the user, and measures biological information on the user. The biological information indicates the psychological state of the user. Examples of the psychological state include a psychological state indicating whether or not the user is feeling satisfied or dissatisfied with use of the image processing apparatus 120. The wearable terminal 110 has the shape of a wristband mounted to an arm, for example, as illustrated in FIG. 1. However, the shape of the wearable terminal 110 is not limited to a wristband shape. For example, the shape of the wearable terminal 110 may be any shape such as the shape of a ring mounted to a finger, the shape of a clip attached to a garment, the shape of glasses mounted to the head, and a head-mount type. The wearable terminal 110 includes one or a plurality of sensors (not illustrated) that measure biological information. The biological information includes a heart rate or a blood pressure, for example. In the case where the biological information includes a heart rate, for example, the wearable terminal 110 includes a heart rate meter that measures the heart rate. In the case where the biological information includes a blood pressure, the wearable terminal 110 includes a sphygmomanometer that measures the blood pressure. The wearable terminal 110 also includes a communication section (not illustrated). The communication section performs data communication with a different device in accordance with Bluetooth (registered trademark), for example. However, the communication standard which is employed by the communication section is not limited to Bluetooth (registered trademark), and may be a different communication standard. The wearable terminal 110 transmits the biological information from the communication section to the image processing apparatus 120, for example.

FIG. 2 illustrates an example of the configuration of the image processing apparatus 120. The image processing apparatus 120 has a plurality of functions to process an image, such as a copy function, a print function, a scan function, and a facsimile function. The image processing apparatus 120 also has a function of requesting the user for a rating related to use of the image processing apparatus 120 after use of the image processing apparatus 120. The image processing apparatus 120 includes a processor 121, a memory 122, a communication section 123, an operation section 124, a display section 125, an image reading section 126, and an image forming section 127. Such sections are connected to each other via a bus 128.

The processor 121 performs a process of controlling the various sections of the image processing apparatus 120 and implementing the functions of the image processing apparatus 120 by executing a program. The processor 121 is a central processing unit (CPU), for example. The memory 122 stores the program for implementing the functions of the image processing apparatus 120. This program includes a copy application program (hereinafter referred to as a “copy app”), a print application program, a scan application program, and a facsimile application program for implementing a copy function, a print function, a scan function, and a facsimile function, respectively. The memory 122 is a read only memory (ROM) and a random access memory (RAM), for example. The memory 122 may also be a hard disk drive or a solid state drive (SSD), for example, in addition to the ROM and the RAM. The communication section 123 performs data communication with a different device connected via a communication line. The communication line may be a wired communication line, or may be a wireless communication line. Examples of the communication standard which is employed by the communication section 123 include Bluetooth (registered trademark). The operation section 124 is used by the user to operate the image processing apparatus 120. Examples of the operation section 124 include a touch screen and buttons. The display section 125 displays various screens that are used to exchange information with the user. The display section 125 is a liquid crystal display, for example. The image reading section 126 reads an image, and converts the image into image data. The image reading section 126 is an image scanner, for example. The image forming section 127 forms an image that matches image data on a medium such as paper. The image forming section 127 is a printer, for example. In the following description, forming an image on a medium is also referred to as “printing”.

2. Operation

In the case where the processor 121 is indicated as the subject of a process in the following description, it is meant that the process is performed by the processor 121 performing computation or controlling operation of other hardware elements through cooperation of the program which is stored in the memory 122 and the processor 121 which executes the program.

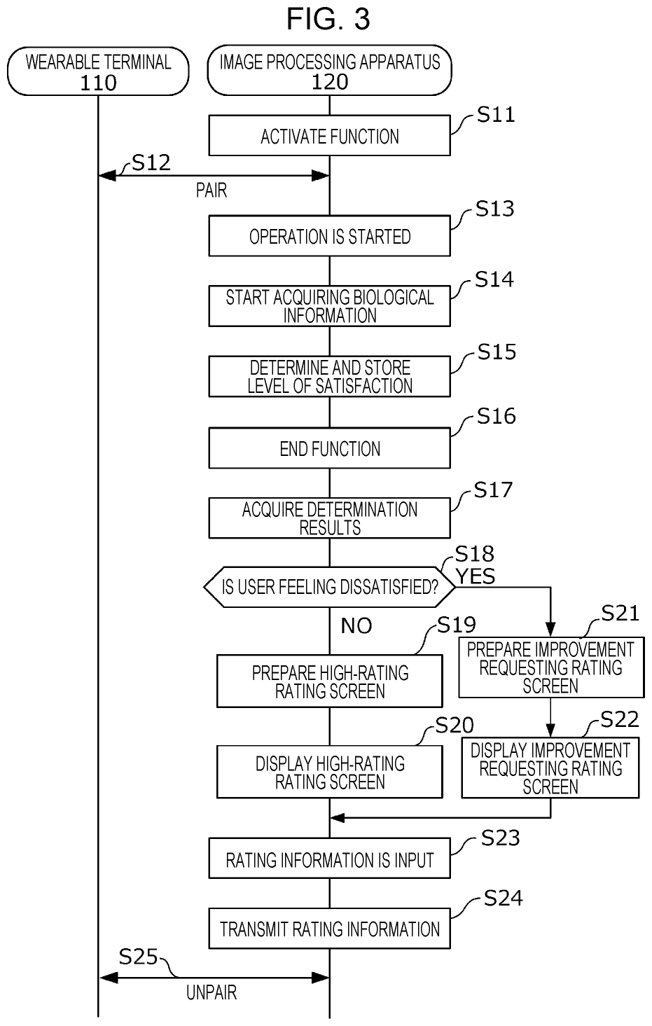

FIG. 3 is a sequence chart illustrating an example of operation performed by the image processing system 100. This operation is performed when the user uses the image processing apparatus 120. In step S11, the processor 121 of the image processing apparatus 120 activates one of the plurality of functions in accordance with an operation by the user.

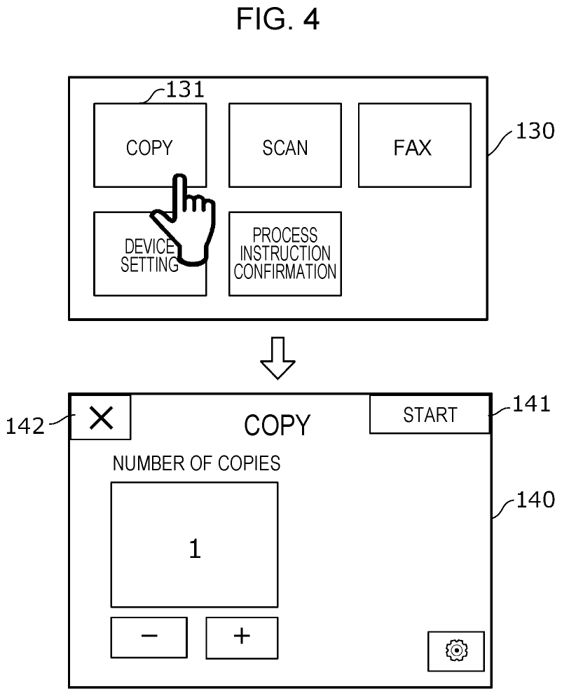

FIG. 4 illustrates an example of screen transition of the display section 125. First, the display section 125 displays a menu screen 130. The menu screen 130 is used by the user to perform an operation to select a desired function from the plurality of functions of the image processing apparatus 120. The menu screen 130 includes a copy button 131 that receives an operation to select the copy function. To select the copy function, the user performs an operation to press the copy button 131 using the operation section 124. When the copy button 131 is pressed, the copy app is executed to activate the copy function.

In step S12, the processor 121 pairs the image processing apparatus 120 with the wearable terminal 110 of the user when the function is started. At this time, first, the wearable terminal 110 of the user is specified. For example, a user ID of the user is correlated with a terminal ID of the wearable terminal 110 of the user in advance. A terminal ID correlated with the user ID which is input at the time of log-in is specified, and the wearable terminal 110 corresponding to the specified terminal ID is specified as the wearable terminal 110 of the user. Subsequently, the image processing apparatus 120 is paired with the specified wearable terminal 110 in accordance with Bluetooth (registered trademark). Consequently, the image processing apparatus 120 and the wearable terminal 110 of the user are wirelessly connected to each other. While the image processing apparatus 120 is paired with one wearable terminal 110, the image processing apparatus 120 is not paired with a different wearable terminal 110.

In step S13, the user starts an operation related to the function which is activated in step S11 using the operation section 124. The processor 121 receives an operation by the user. In the example illustrated in FIG. 4, when the user performs an operation to press the copy button 131, a copy screen 140 is displayed on the display section 125. The user performs an operation to set various setting values related to the copy function using the operation section 124 on the copy screen 140.

In step S14, the processor 121 acquires biological information from the wearable terminal 110 when the user starts the operation. The biological information is acquired at time intervals determined in advance. Specifically, the processor 121 transmits, from the communication section 123, a request to acquire biological information to the wearable terminal 110 which is paired in step S12. The wearable terminal 110 transmits biological information measured from the user to the image processing apparatus 120 in response to this acquisition request. The wearable terminal 110 receives, at the communication section 123, the biological information which is transmitted from the wearable terminal 110.

In step S15, the processor 121 determines the level of satisfaction of the user with the function that is activated, by analyzing biological information each time such biological information is acquired from the wearable terminal 110, and stores the determination results in the memory 122. This determination is made in accordance with whether or not the biological information indicates that the user is feeling dissatisfied. It is assumed that the biological information includes a heart rate or a blood pressure. When the amount of increase in the heart rate or the blood pressure is equal to or more than a threshold, for example, it is indicated that the user is feeling dissatisfied, and therefore it is determined that the level of satisfaction is low. The threshold value is set in advance to a minimum value of the amount of increase in the heart rate or the blood pressure measured when a person is feeling dissatisfied, for example. In the case where the amount of increase in the heart rate or the blood pressure is less than the threshold, on the other hand, it is indicated that the user is not feeling dissatisfied, and therefore it is determined that the level of satisfaction is high.

In step S16, the processor 121 ends the function that is activated when processing of such a function is completed. In the example illustrated in FIG. 4, the copy screen 140 includes a start button 141 that receives an operation to provide an instruction to start a copy process. When the user performs an operation to press the start button 141, the copy process is executed in accordance with setting values set in accordance with an operation by the user. In the copy process, the image reading section 126 reads an image of a document and converts the image into image data, and the image forming section 127 forms an image that matches the image data on a medium.

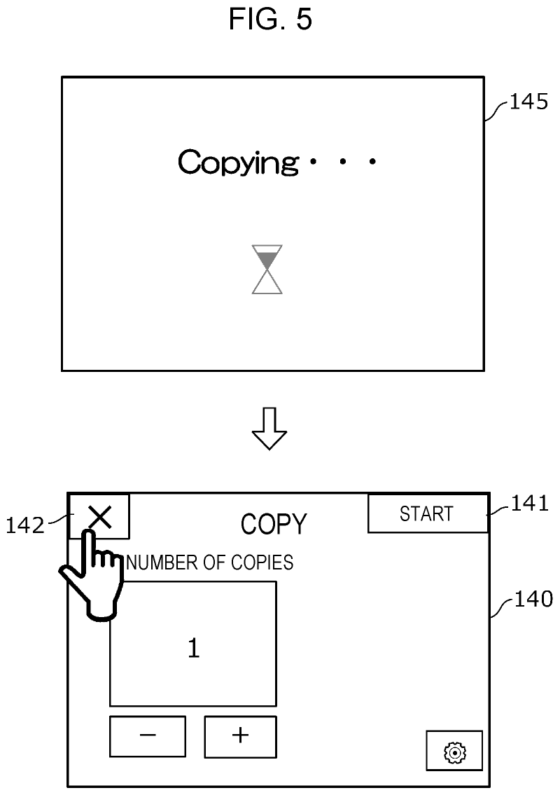

FIG. 5 illustrates an example of screen transition of the display section 125. While the copy process is performed, the display section 125 displays a notification screen 145 indicating that the process is being performed as illustrated in FIG. 5. When the copy process is completed, the display section 125 displays the copy screen 140 again as illustrated in FIG. 5. The copy screen 140 includes a close button 142 that receives an operation to end the copy function. When the user performs an operation to press the close button 142 included in the copy screen 140 using the operation section 124, the copy app is stopped, and the copy function is ended.

In step S17, the processor 121 acquires the results of the determination in step S15 from the memory 122. At this time, acquisition of biological information from the wearable terminal 110 may be ended. That is, biological information is acquired at determined time intervals continuously at least for a period for which the user uses the function. The period for which the user uses the function includes a period for which the user performs an operation related to the function that is activated and a period for which processing of the function that is activated is performed.

In step S18, the processor 121 determines whether or not the user is feeling dissatisfied with the function that he/she used on the basis of the determination results which are acquired in step S17. In the case where all of the determination results stored in the memory 122 indicate that the level of satisfaction of the user is high, for example, it is determined that the user is not feeling dissatisfied with the function that he/she used (NO in the determination in step S18). In this case, the processor 121 proceeds to the process in step S19.

In step S19, the processor 121 prepares a high-rating rating screen that is used for an operation to rate the function used by the user. The high-rating rating screen is a screen that is suitable for a user who is not feeling dissatisfied with the function that he/she used to perform an operation to give a rating related to use of the function. In step S20, the processor 121 performs control so as to display the high-rating rating screen prepared in step S19 on the display section 125.

In the case where at least one of the determination results stored in the memory 122 indicates that the level of satisfaction of the user is low, for example, in step S18 discussed above, on the other hand, it is determined that the user is feeling dissatisfied with the function that he/she used (YES in the determination in step S18). In this case, the processor 121 proceeds to the process in step S21.

In step S21, the processor 121 prepares an improvement requesting rating screen that is used for an operation to rate the function used by the user. The improvement requesting rating screen is a screen that is suitable for a user who is feeling dissatisfied with the function that he/she used to perform an operation to give a rating requesting an improvement of the function. In step S22, the processor 121 performs control so as to display the improvement requesting rating screen prepared in step S21 on the display section 125. In this manner, in this exemplary embodiment, the processor 121 makes the rating screen different in accordance with whether or not the user is feeling dissatisfied with the function that he/she used.

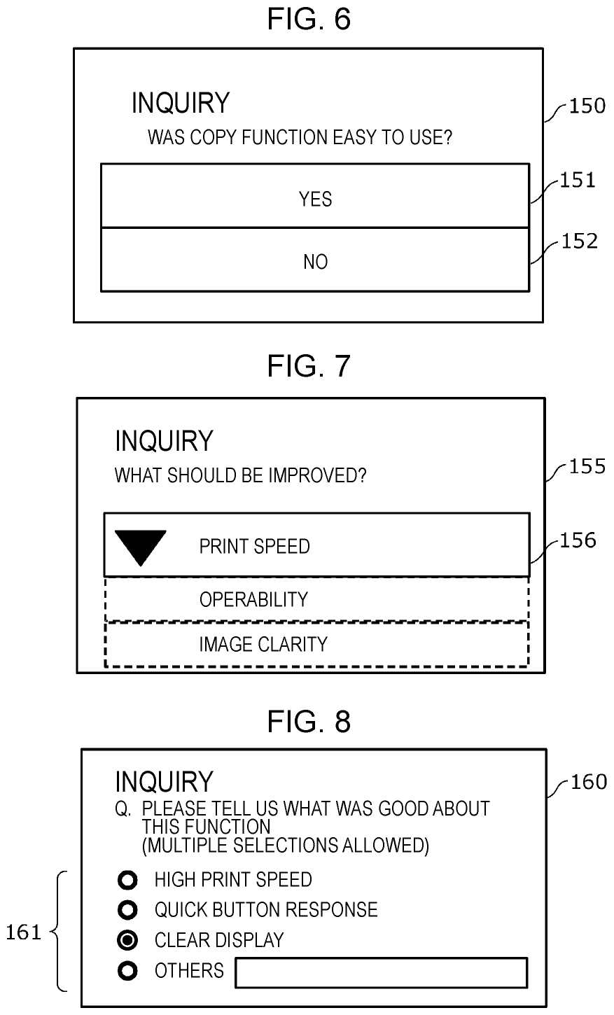

FIG. 6 illustrates a high-rating rating screen 150 according to a first exemplary embodiment. FIG. 7 illustrates an improvement requesting rating screen 155 according to the first exemplary embodiment. A case where the user uses the copy function is assumed. In the high-rating rating screen 150, the user is not requested to give a detailed rating answering what specifically about the function was easy to use, but is requested to give only a simple rating answering whether or not the function was easy to use. This is because it is highly likely that the user does not have a request for an improvement of the function in the case where the user is not feeling dissatisfied with the function that he/she used and, therefore, it is considered to be good enough to simply inquire of the user about whether or not the function was easy to use. In the improvement requesting rating screen 155, the user is requested to give a detailed rating answering what specifically about the function is to be improved, rather than a simple rating answering whether or not there is anything about the function to be improved. This is because it is necessary to specifically inquire about what specifically about the function is requested to be improved in the case where the user is feeling dissatisfied with the function that he/she used. In the first exemplary embodiment, the high-rating rating screen 150 and the improvement requesting rating screen 155 are different from each other in the question related to the rating, the options of answers related to the rating, and the number of options related to the rating.

As illustrated in FIG. 6, the high-rating rating screen 150 includes a question “WAS COPY FUNCTION EASY TO USE?”. This is because it is considered to be highly possible that the user is feeling that the copy function was easy to use in the case where the user is not feeling dissatisfied with use of the copy function. In the high-rating rating screen 150, a choice type is adopted as the manner of answering to the question. The choice type refers to the manner of answering by selecting one of a plurality of options. The high-rating rating screen 150 includes a “YES” button 151 that receives an operation to give a positive answer to the question, and a “NO” button 152 that receives an operation to give a negative answer to the question. That is, the high-rating rating screen 150 includes two options of answers, namely a positive answer and a negative answer. This is intended to allow giving a rating with a simple operation, since it is considered to be less likely that the user cooperates in giving a rating which takes much trouble, in the case where the user is not feeling dissatisfied with use of the copy function.

On the other hand, as illustrated in FIG. 7, the improvement requesting rating screen 155 includes a question “WHAT SHOULD BE IMPROVED?”. This is because it is considered that the user has a specific request about what about the copy function is to be improved in the case where the user is feeling dissatisfied with use of the copy function. In the improvement requesting rating screen 155, a choice type is adopted as the manner of answering to the question. The improvement requesting rating screen 155 includes a select list 156. The select list 156 includes a plurality of candidates for points to be improved assumed in relation to the copy function, such as “PRINT SPEED”, “OPERABILITY”, and “IMAGE CLARITY”. The select list 156 receives an operation to select what the user desires to be improved, from among the candidates for points to be improved. That is, the improvement requesting rating screen 155 includes three or more options of answers including “PRINT SPEED”, “OPERABILITY”, and “IMAGE CLARITY”. This is intended to obtain detailed rating information, since it is considered to be likely that the user cooperates in giving a rating in order for an improvement, in the case where the user is feeling dissatisfied with use of the copy function. The plurality of candidates for points to be improved assumed in relation to each function are determined in advance.

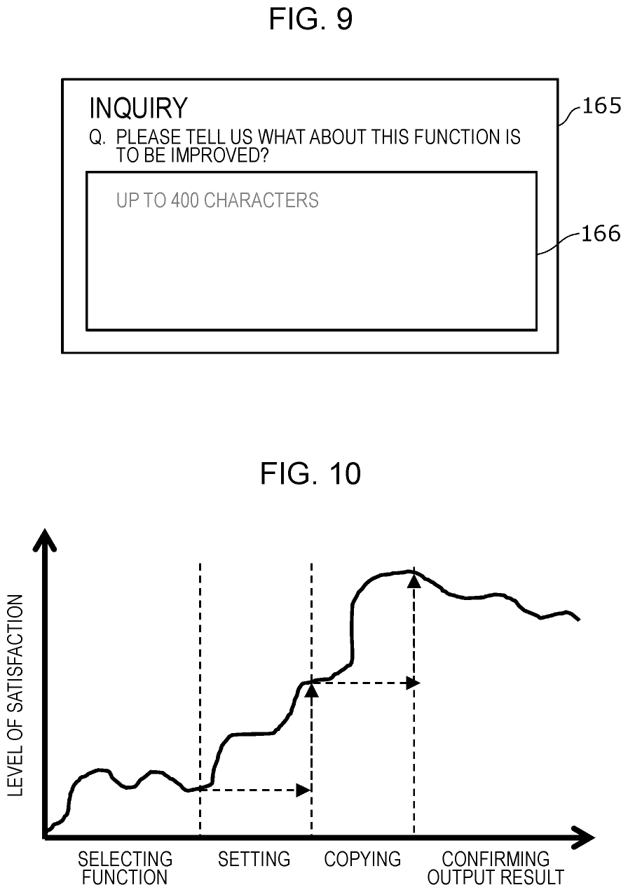

FIG. 8 illustrates a high-rating rating screen 160 according to a second exemplary embodiment. FIG. 9 illustrates an improvement requesting rating screen 165 according to the second exemplary embodiment. A case where the user uses the copy function is assumed. In each of the high-rating rating screen 160 and the improvement requesting rating screen 165, the user is requested to give a detailed rating answering what specifically is good and what specifically is to be improved. However, the improvement requesting rating screen 165 offers a higher degree of freedom in answering than the high-rating rating screen 160. This is intended to obtain detailed rating information, since it is considered to be likely that the user cooperates in giving a rating in order for an improvement, in the case where the user is feeling dissatisfied with use of the copy function. In the second exemplary embodiment, the high-rating rating screen 160 and the improvement requesting rating screen 165 adopt different manners of answering for a rating.

As illustrated in FIG. 8, the high-rating rating screen 160 includes a question “PLEASE TELL US WHAT WAS GOOD ABOUT THIS FUNCTION”. In the high-rating rating screen 160, a choice type is adopted as the manner of answering to the question. The high-rating rating screen 160 includes a plurality of select buttons 161. The plurality of select buttons 161 correspond to a plurality of candidates for good points assumed in relation to the copy function, such as “high print speed”, “quick button response”, and “clear display”. The plurality of select buttons 161 receive an operation by the user to select a good point desired by the user, from among the candidates for good points. The plurality of candidates for good points assumed in relation to each function are determined in advance.

On the other hand, as illustrated in FIG. 9, the improvement requesting rating screen 165 includes a question “PLEASE TELL US WHAT ABOUT THIS FUNCTION IS TO BE IMPROVED”. In the improvement requesting rating screen 165, a free writing type is adopted as the manner of answering to the question. The improvement requesting rating screen 165 includes an input region166. The input region 166 receives an operation to input characters, numerals, and symbols. The user may freely write what specifically about the copy function is to be improved in the input region 166.

Returning to FIG. 3, in step S23, the user performs an operation to input rating information on the rating screen displayed on the display section 125 in step S20 or S22 using the operation section 124. The processor 121 receives this operation. For example, when the user performs an operation to press the “YES” button 151 using the operation section 124 in the case where the high-rating rating screen 150 illustrated in FIG. 6 is displayed, rating information indicating that the copy function was easy to use is input. When the user performs an operation to select “PRINT SPEED” from the select list 156 using the operation section 124 in the case where the improvement requesting rating screen 155 illustrated in FIG. 7 is displayed, on the other hand, rating information indicating that the print speed should be improved is input.

In step S24, the processor 121 transmits the rating information input in step S23 from the communication section 123 to a transmission destination determined in advance. This transmission destination may be a server device connected to the image processing apparatus 120 via a communication line such as the Internet, for example. In step S25, the processor 121 unpairs the image processing apparatus 120 from the wearable terminal 110.

3. Modifications

The exemplary embodiments discussed above are examples of the present disclosure. The present disclosure is not limited to the exemplary embodiments discussed above. The exemplary embodiments discussed above may be modified as in the following examples to be implemented. Two or more of the following modifications may be combined with each other.

3.1 Modification 1

In the exemplary embodiments discussed above, the options of answers included in the rating screen may be different in accordance with the level of satisfaction of the user during processes performed while a function is activated. In this case, the processor 121 determines the level of satisfaction of the user for each of the processes performed while the function is activated. These processes may be separated for each screen displayed on the display section 125, for example.

FIG. 10 illustrates an example of the level of satisfaction of the user during processes performed while the copy function is activated. In FIG. 10, the horizontal axis indicates the flow of processes performed while the copy function is activated, and the vertical axis indicates the level of satisfaction of the user. The processes performed while the copy function is activated include “selecting function”, “setting”, “copying”, and “confirming output result” processes. In the “selecting function” process, the user selects the copy function on the menu screen 130 illustrated in FIG. 4. In the “setting” process, the user sets various setting values related to the copy function on the copy screen 140 illustrated in FIG. 4, for example. In the “copying” process, a copy process is performed. As discussed above, the notification screen 145 illustrated in FIG. 5 is displayed during this process. In the “confirming output result” process, the user confirms the output result of the copy process. As illustrated in FIG. 5, the copy screen 140 is displayed during this process. The level of satisfaction of the user is expressed in multiple, three or more, levels in accordance with the biological information on the user. For example, the level of satisfaction is lower as the amount of increase in the blood pressure or the heart rate is larger. In the example illustrated in FIG. 10, the amount of increase in the level of satisfaction of the user is equal to or more than a threshold in the “setting” and “copying” processes. This indicates that the level of satisfaction of the user is high in such processes. The state in which “the level of satisfaction of the user is high” is an example of the “specific psychological state” according to the present disclosure.

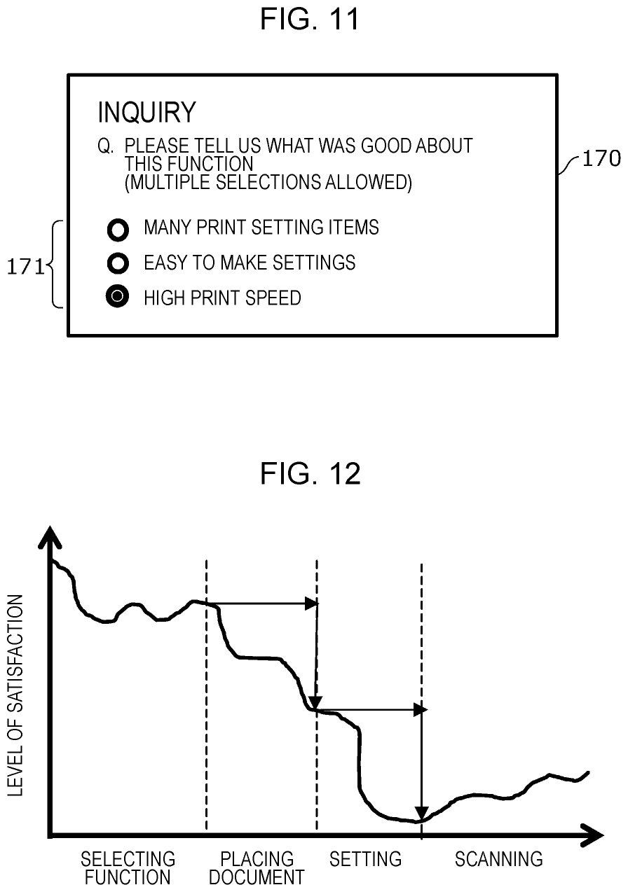

FIG. 11 illustrates an example of a high-rating rating screen 170 prepared and displayed in the example illustrated in FIG. 10. The high-rating rating screen 170 includes a plurality of select buttons 171. The plurality of select buttons 171 correspond to candidates for a good point “MANY PRINT SETTING ITEMS” and “EASY TO MAKE SETTINGS” assumed for the “setting” process with which the level of satisfaction of the user is high, and a candidate for a good point “HIGH PRINT SPEED” assumed for the “copying” process. The plurality of select buttons 171 receive an operation by the user to select a desired good point, from among the candidates for good points. That is, the high-rating rating screen 170 includes options of answers related to the rating of the “setting” process and the “copying” process performed when the user was feeling satisfied. The candidates for good points for each process are determined in advance. Meanwhile, the high-rating rating screen 170 does not include options of answers related to the rating of the “selecting function” and “confirming output result” processes. That is, in the high-rating rating screen 170, options of answers related to the rating of the “setting” and “copying” processes performed when the user was feeling satisfied are displayed in priority to options of answers related to the rating of the other processes. The “options of answers related to the rating of the other processes” are an example of the “other options” according to the present disclosure.

FIG. 12 illustrates an example of the level of satisfaction of the user during processes performed while the scan function is activated. In FIG. 12, the horizontal axis indicates the flow of processes performed while the scan function is activated, and the vertical axis indicates the level of satisfaction of the user. The processes performed while the scan function is activated include “selecting function”, “placing document”, “setting”, and “scanning” processes. In the “selecting function” process, as in the “selecting function” process of the copy function, the user selects the scan function. In the “placing document” process, the user places a document on the image reading section 126. In the “setting” process, as in the “setting” process of the copy function, the user sets various setting values related to the scan function. In the “scanning” process, a scan process is performed. The level of satisfaction of the user is expressed in multiple, three or more, levels in accordance with the biological information on the user, as in the example illustrated in FIG. 10. In the example illustrated in FIG. 12, the amount of decrease in the level of satisfaction of the user is equal to or more than a threshold in the “placing document” and “setting” processes. This indicates that the level of satisfaction of the user is low in such processes. The state in which “the level of satisfaction of the user is low” is an example of the “specific psychological state” according to the present disclosure.

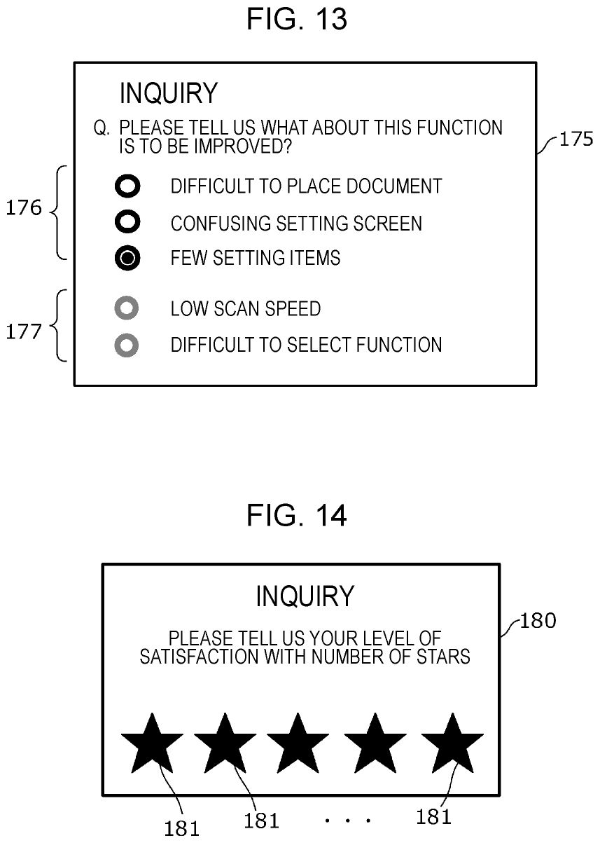

FIG. 13 illustrates an example of an improvement requesting rating screen 175 prepared and displayed in the example illustrated in FIG. 12. The improvement requesting rating screen 175 includes a plurality of select buttons 176. The plurality of select buttons 176 correspond to a candidate for a point to be improved “DIFFICULT TO PLACE DOCUMENT” assumed for the “placing document” process with which the level of satisfaction of the user is low, and candidates for points to be improved “CONFUSING SETTING SCREEN” and “FEW SETTING ITEMS” assumed for the “setting” process. The plurality of select buttons 176 receive an operation to select what the user desires to be improved, from among the candidates for points to be improved. That is, the improvement requesting rating screen 175 includes options of answers related to the rating of the “placing document” and “setting” processes performed when the user was feeling dissatisfied. The candidates for points to be improved for each process are determined in advance. The improvement requesting rating screen 175 also includes a plurality of select buttons 177. The plurality of select buttons 177 correspond to a candidate for a point to be improved “DIFFICULT TO SELECT FUNCTION” assumed for the rating of the “selecting function” process, and a candidate for a point to be improved “LOW SCAN SPEED” assumed for the rating of the “scanning” process. The plurality of select buttons 177 receive an operation to select what the user desires to be improved, from among the candidates for points to be improved. The candidates for points to be improved for each process are determined in advance. In the improvement requesting rating screen 175, however, options of answers related to the rating of the “placing document” and “setting” processes performed when the user was feeling dissatisfied are displayed in priority to options of answers related to the rating of the other processes. The “options of answers related to the rating of the other processes” are an example of the “other options” according to the present disclosure. For example, the plurality of select buttons 177 are displayed below the plurality of select buttons 176. That is, the plurality of select buttons 176 are displayed above the plurality of select buttons 177. In addition, the plurality of select buttons 177 may be displayed in an indistinctive color compared to the plurality of select buttons 176. That is, the plurality of select buttons 176 may be displayed in a distinctive color compared to the plurality of select buttons 177. The method of displaying in priority is not limited to displaying above and displaying in a distinctive color. For example, the size of the target options may be increased, an additional image may be added to the target options, or the target options may be displayed in an exaggerating manner.

3.2 Modification 2

In the exemplary embodiments discussed above, the user may be requested to give a detailed rating answering what specifically about the function was easy to use in the high-rating rating screen, and the user may be requested to give a simple rating answering whether or not there is anything about the function to be improved in the improvement requesting rating screen, as opposed to the first exemplary embodiment and the second exemplary embodiment discussed above, for example. This is intended to allow giving a rating with a simple operation, since it is considered to be less likely that the user cooperates in giving a rating which takes much trouble, in the case where the user is feeling dissatisfied with use of the function that he/she used, in the case where the purpose of use of the rating information is to investigate the level of satisfaction of the user or to investigate the function to be developed next, for example. It is also intended to obtain detailed rating information, on the other hand, since it is considered to be likely that the user cooperates in giving a rating, in the case where the user is not feeling dissatisfied with the function that he/she used. For example, a free writing type may be adopted as the manner of answering to the question in the high-rating rating screen, and a choice type may be adopted as the manner of answering to the question in the improvement requesting rating screen.

3.3 Modification 3

In the exemplary embodiments discussed above, in the case where a choice type is adopted as the manner of answering to the question in the high-rating rating screen or the improvement requesting rating screen, the option selected in advance among a plurality of options may be different in accordance with whether or not the user is feeling dissatisfied with the function that he/she used. In addition, the number of rating screens may be different in accordance with whether or not the user is feeling dissatisfied with the function that he/she used. FIG. 14 illustrates an example of a high-rating rating screen 180 according to such a modification. The high-rating rating screen 180 includes five star-shaped select buttons 181. These select buttons 181 receive an operation to select stars. It is indicated that the level of satisfaction of the user is higher as the number of selected starts is larger. In the high-rating rating screen 180, five stars are selected in advance. This is because it is considered to be highly possible that the user selects five stars, since the level of satisfaction is high, in the case where the user is not feeling dissatisfied with use of the function that he/she used. In the case where the number of stars according to the level of satisfaction is not five, the user performs an operation to press the select button 181 so as to select the number of starts according to the level of satisfaction using the operation section 124.

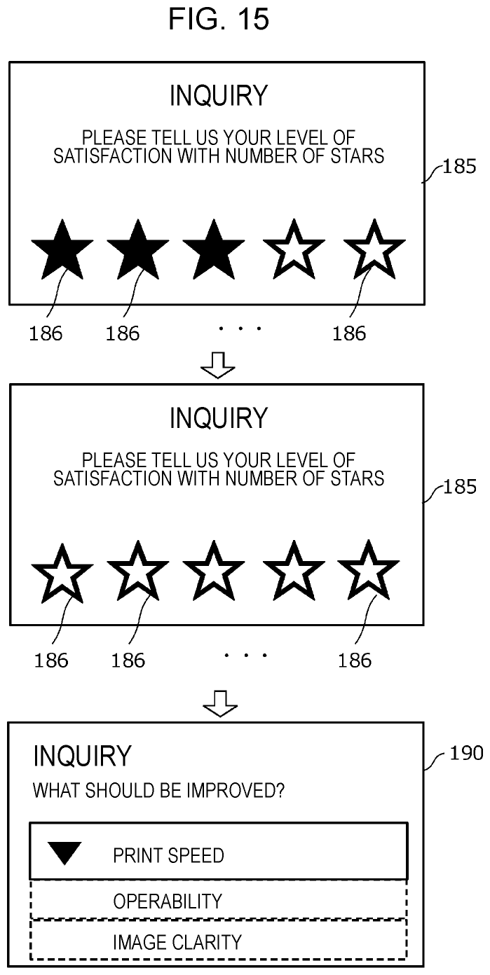

FIG. 15 illustrates an example of an improvement requesting rating screen185 according to the modification. The improvement requesting rating screen 185 includes five star-shaped select buttons 186, as with the high-rating rating screen 180 illustrated in FIG. 14. In the improvement requesting rating screen 185, however, the number of starts selected in advance is smaller than that in the high-rating rating screen 180. In the improvement requesting rating screen 185, for example, three stars are selected in advance. This is because it is considered to be highly possible that the user selects less than five stars, since the level of satisfaction is low, in the case where the user is feeling dissatisfied with use of the function that he/she used. In the case where the number of stars according to the level of satisfaction is not three, the user performs an operation to press the select button 186 so as to select the number of starts according to the level of satisfaction using the operation section 124.

In the case where the number of stars selected by the user in the improvement requesting rating screen 185 is equal to or less than a threshold, a detailed improvement requesting rating screen 190 is further displayed as illustrated in FIG. 15. This threshold is set in advance to a number indicating that the level of satisfaction of the user is low. It is assumed that the threshold is two. As illustrated in FIG. 15, when the user performs an operation to press the select button 186 corresponding to three stars selected in advance, the select button 186 is unselected, and the number of selected stars becomes zero. In this case, the number of stars selected by the user is equal to or less than the threshold, and therefore the detailed improvement requesting rating screen 190 is further displayed as illustrated in FIG. 15. In the detailed improvement requesting rating screen 190, as in the improvement requesting rating screen 155 illustrated in FIG. 7, the user is requested to give a detailed rating answering what specifically is to be improved. In this modification, in this manner, only one rating screen 180 is displayed as illustrated in FIG. 14 in the case where the user is not feeling dissatisfied with the function that he/she used, while two rating screens 185 and 190 are displayed in the case where the user is feeling dissatisfied with the function that he/she used.

3.4 Modification 4

In the exemplary embodiments discussed above, the psychological state of the user is not limited to whether or not he/she is feeling dissatisfied with the function that he/she used. For example, the psychological state of the user may be a psychological state indicating whether or not he/she is in a rush or whether or not he/she is feeling stressed. A rating screen that puts an early end to input of rating information may be displayed in the case where the user is in a rush or feeling stressed, for example. Examples of the rating screen that puts an early end to input of rating information include a rating screen in which a choice type is adopted as the manner of answering to the question and a rating screen with a smaller number of options such as the rating screen 150 illustrated in FIG. 6. On the other hand, a rating screen through which detail rating information is obtainable may be displayed in the case where the user is not in a rush or not feeling stressed. Examples of the rating screen through which detail rating information is obtainable include a rating screen with a large number options of answers such as the rating screen 155 illustrated in FIG. 7 and a rating screen in which a free writing type is adopted as the manner of answering to the question such as the rating screen 165 illustrated in FIG. 9.

3.5 Modification 5

In the exemplary embodiments discussed above, the threshold for the biological information that is used to determine the level of satisfaction may be changed in accordance with the purpose of use of the rating information. For example, in the case where the purpose of use of the rating information is to investigate a point of the function to be improved, the threshold for the biological information that is used to determine the level of satisfaction may be reduced. In the case where the purpose of use of the rating information is to investigate the level of satisfaction of the user, on the other hand, the threshold for the biological information that is used to determine the level of satisfaction may be increased.

3.6 Modification 6

In the exemplary embodiments discussed above, the rating screen may be displayed for only some of the functions of the image processing apparatus 120. For example, the image processing apparatus 120 occasionally includes a process instruction confirmation function, besides the print function, the copy function, the scan function, and the facsimile function. The process instruction confirmation function is a function to allow the user to confirm the status of a process instruction provided to the image processing apparatus 120, the status of progress of a process, and a history of processes. Such a function normally does not require a rating by the user. Thus, the process for displaying the rating screen discussed above may not be performed in the case where such a function is activated on the menu screen 130 illustrated in FIG. 4, for example.

3.7 Modification 7

In the exemplary embodiments discussed above, the information processing apparatus according to the present disclosure is not limited to the image processing apparatus 120. The information processing apparatus may be any device that is used by the user and that obtains rating information related to use from the user, such as a smartphone, a tablet terminal, a personal computer, a home electric appliance, an audio device, and a gaming device. A case where the information processing apparatus is a smartphone connected to the image processing apparatus 120 via a wireless communication line, for example, is assumed. In this case, the information processing apparatus is paired with the wearable terminal 110 when the user performs an operation to select an icon on a menu screen displayed on the smartphone, for example. Acquisition of biological information is started when an application program is executed in response to this operation, for example. This application program may be a program for implementing a function to provide the image processing apparatus 120 with an instruction for a print process, for example. The determination as to whether or not the user is feeling dissatisfied with use of the application program is performed when the application program is stopped, for example. In the case where the user is not feeling dissatisfied with use of the application program, the high-rating rating screen 150 or 160 illustrated in FIG. 6 or 8, for example, is displayed on the smartphone. In the case where the user is feeling dissatisfied with use of the application program, on the other hand, the improvement requesting rating screen 155 or 165 illustrated in FIG. 7 or 9, for example, is displayed on the smartphone.

3.8 Modification 8

The subjects of the processes in the image processing system 100 according to the exemplary embodiments discussed above are exemplary, and are not limited to such examples. For example, at least some of the processes performed by the image processing apparatus 120 may be performed by a different device. In addition, the processes in the image processing system 100 according to the exemplary embodiments discussed above are exemplary, and may be reordered unless any contradiction occurs.

3.9 Modification 9

In the embodiments above, the term “processor” refers to hardware in a broad sense. Examples of the processor include general processors (e.g., CPU: Central Processing Unit) and dedicated processors (e.g., GPU: Graphics Processing Unit, ASIC: Application Specific Integrated Circuit, FPGA: Field Programmable Gate Array, and programmable logic device).

In the embodiments above, the term “processor” is broad enough to encompass one processor or plural processors in collaboration which are located physically apart from each other but may work cooperatively. The order of operations of the processor is not limited to one described in the embodiments above, and may be changed.

3.10 Modification 10

The present disclosure may be provided as a program to be executed by each of the image processing apparatus 120 and the wearable terminal 110. The image processing apparatus 120 and the wearable terminal 110 are each an example of the computer according to the present disclosure. The program may be downloaded via a communication line such as the Internet, and may be provided as recorded in a computer-readable recording medium such as a magnetic recording medium (such as a magnetic tape and a magnetic disk), an optical recording medium (such as an optical disc), a magneto-optical recording medium, and a semiconductor memory.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a processor configured to
acquire biological information on a user while the user is using the information processing apparatus,
perform control so as to display, on a display section, a rating screen that is used to perform an operation to give a rating related to use of the information processing apparatus, and
perform control so as to display, on the display section, different rating screens in accordance with a psychological state of the user indicated by the biological information.

2. The information processing apparatus according to claim 1,
wherein options of answers related to the rating included in the rating screen are different in accordance with the psychological state.

3. The information processing apparatus according to claim 2,
wherein the rating screen includes options of answers related to a rating of a process performed when the user is brought into a specific psychological state.

4. The information processing apparatus according to claim 3,
wherein the processor displays the options in priority to other options.

5. The information processing apparatus according to claim 2,
wherein a number of the options is different in accordance with the psychological state.

6. The information processing apparatus according to claim 1,
wherein an option selected in advance, of a plurality of options of answers related to the rating included in the rating screen, is different in accordance with the psychological state.

7. The information processing apparatus according to claim 1,
wherein a manner of answering for the rating on the rating screen is different in accordance with the psychological state.

8. The information processing apparatus according to claim 1,
wherein a question related to the rating included in the rating screen is different in accordance with the psychological state.

9. The information processing apparatus according to claim 1,
wherein a number of rating screens is different in accordance with the psychological state.

10. A non-transitory computer readable medium storing a program causing a computer to execute a process comprising:
acquiring biological information on a user while the user is using an apparatus,
performing control so as to display, on a display section, a rating screen that is used to perform an operation to give a rating related to use of the apparatus, and
performing control so as to display, on the display section, different rating screens in accordance with a psychological state of the user indicated by the biological information.

11. An information processing apparatus comprising:
means for acquiring biological information on a user while the user is using the apparatus,
means for performing control so as to display, on a display section, a rating screen that is used to perform an operation to give a rating related to use of the information processing apparatus, and
means for performing control so as to display, on the display section, different rating screens in accordance with a psychological state of the user indicated by the biological information.
